(19) United States
(12) Patent Application Publication
(10) Pub. No.: US 2015/0018828 A1
(43) Pub. Date: Jan. 15, 2015

(54) Anti-Septic Transarticular Intramedullary Rod System for the Human Knee

(71) Applicant: John R. Dorris, Athens, GA (US)

(72) Inventor: John R. Dorris, Athens, GA (US)

(21) Appl. No.: 14/264,310

(22) Filed: Apr. 29, 2014

Related U.S. Application Data
(60) Provisional application No. 61/817,449, filed on Apr. 30, 2013.

(51) Int. Cl. A61F 2/38; A61B 17/02

(52) U.S. Cl. CPC A61F 2/3836; A61B 17/025; A61F 2002/30672
USPC 606/62

(57) ABSTRACT

A temporary knee implant to be placed at the position of the human knee joint for maintaining the tibia and femur at the knee joint aligned during healing of infection in the surrounding tissue. An elongated femur rod is formed with one end of a breadth sized and shaped to extend into the medullary cavity of the femur of a knee, and a connector at the other end, an elongated tibia rod of a breadth sized and shaped to extend into the medullary cavity of the tibia and a connector at the other end, the connectors including interfitting shapes for aligning said femur rod and said tibia rod, and medication adhered to the femur and tibia rods for engagement with the medullary surfaces.

### PRIORITY

Priority is claimed to U.S. Provisional Patent Application Ser. No. 61/817,449, filed in the U.S. Patent & Trademark Office on Apr. 30, 2013.

### FIELD OF THE INVENTION

This disclosure concerns the method and apparatus for maintaining a joint alignment and spacing of a knee of the human body during recovery from infected knee prosthesis replacement.

### BACKGROUND OF THE DISCLOSURE

Approximately 800,000 primary total knee replacements are performed in the USA annually. This number is expected to be 1.5 million knee replacements by the year 2020 and 3.5 million knee replacements per year by the year 2030.

Recent publications have indicated a knee sepsis rate of three percent in primary total knee replacements (“TKRS”) and 12 to 16 percent in revision of TKRS. This indicates that over 20,000 surgical procedures for septic prosthetic knees are performed annually.

The procedure for replacing the sepsic knee prosthesis usually is a two stage procedure. Stage one includes the removal of the implanted prosthesis from the infected knee joint, clean the area, inserting a temporary implant in the infected knee during the infection recovery, and after the infection has been eradicated, removing the temporary implant and inserting a revision replacement knee implant. It is estimated that by the year 2020 there will be about 45,000 revisions for knee sepsis of knee prosthesis performed per year.

Generally, there are two types of stage one implants, articulating spacers and static spacers. The articulating spacers allow the knee to move while the static spacers maintain the leg in its extended position. Recent studies have suggested that articulating spacers are not superior to static spacers. It has been reported that articulating spacers increase bone loss due to range of motion and weight bearing. A known complication of static spacers is subluxation. Approximately twelve percent of patients fail current treatment for prosthetic knee sepsis, with above-the-knee amputation sometimes being required.

### SUMMARY OF THE INVENTION

The transarticular intramedullary rod system disclosed herein is intended to supply the surgeon with a very stable static spacer system for the human leg to maintain the joint space of the knee after the initial knee prosthesis has become infected and removed, and before the replacement prosthesis is inserted.

The proposed method and apparatus increases the delivery of antibiotic to the infected bone and essentially eliminates subluxation risk during a two staged procedure. It also allows the surgeon to customize antibiotic type and concentration in the spacer system. It redistributes the compressive forces to the articular surfaces to the more proximal and distal cortical bone. It is an excellent treatment option for infected total knee replacements “TKRS” with extensor mechanism injuries, some of which are ultimately treated with above the knee amputation. Also, it's a much more comfortable scenario for the patient during the two months that is typically needed to eradicate the infection by reducing recurrent fatigue associated standard spacer utilization.

### BRIEF DESCRIPTION OF THE DRAWINGS

FIGS. 1-3 are cross sectional views of the anti-septic transarticular intramedullary rod system extending from the femur into the tibia, showing in FIG. 1 the anterior view, in

FIG. 2 the medial view and in

FIG. 3 the posterior view.

FIG. 4 is a side view of the rods of the implant with its femur rod aligned with the tibia rod and the connector collars attached to each other.

FIG. 5 is a close up side cross sectional view of the collar connectors as shown in the circle of FIG. 4, showing how the collars of the collar connectors become juxtaposed when connected together.

FIG. 6 is a side view of the implant, similar to FIG. 4, but showing the PMMA mantle applied to the rods.

FIG. 7 is a detail view of the collar connectors as shown in the circle of FIG. 6.

### DETAILED DESCRIPTION

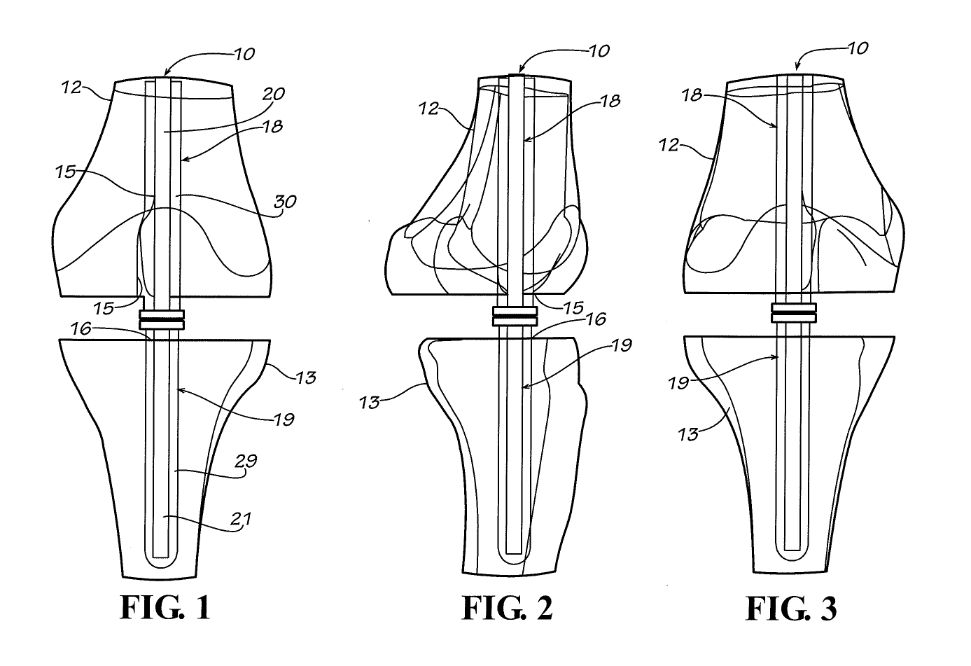

Referring now in more detail to the drawings in which like numerals indicate like parts throughout the several views, FIGS. 1-3 disclose a temporary knee implant 10 as it would be inserted at the location of the knee joint, between the femur 12 and the tibia 13. The three drawings show the anterior, medial and posterior views, respectively, of the position of the human body. The temporary knee implant is inserted in the medullary cavities 15 and 16 of the femur and tibia, respectively.

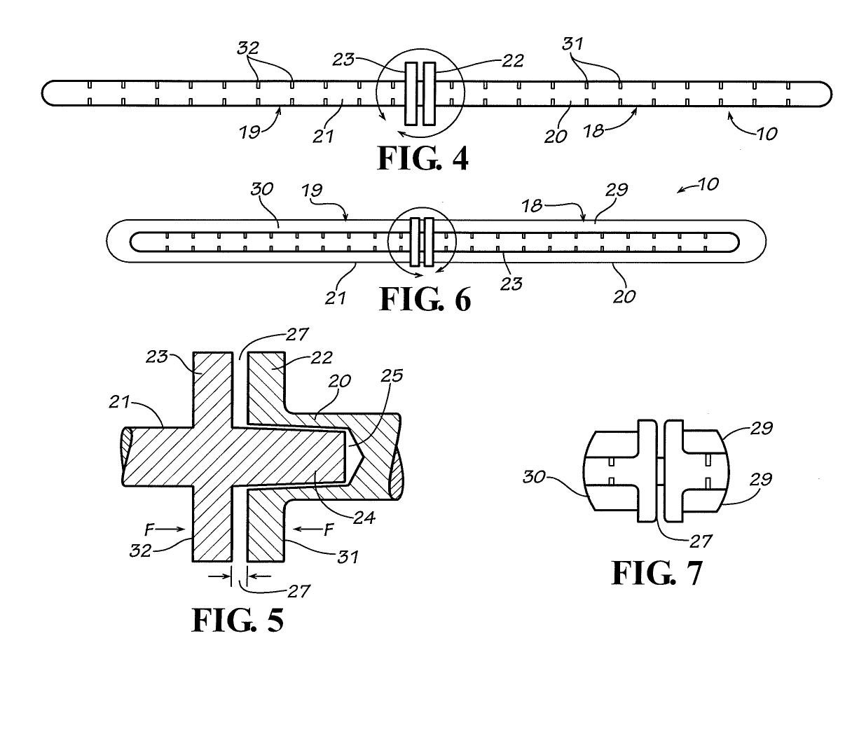

As best shown in FIG. 4, the temporary knee implant 10 includes an elongated femur rod 18 and a similar elongated tibia rod 19. The femur rod includes a femur stem 20 and the tibia rod includes its tibia stem 21 and both include connector collars 22 and 23, respectively. The femur connector collar 22 is mounted to the distal end of the femur stem 20, and the tibia connector collar 23 is connected to the proximal end of the tibia stem. The collars 22 and 23 are disk-shaped, but may be of other shapes that provide similar functions.

As illustrated in FIG. 5, the femur connector collar 22 and the tibia connector collar 23 include complementary connectors formed by interfitting shapes for aligning the femur stem 20 and the tibia stem 21. The interfitting shapes may include a tapered femur locking pin 24 that protrudes beyond the femur connector collar 23, and a tibia socket 25 formed in the tibia connector collar 23 that is tapered and sized and shaped to receive the femur locking pin 24.

When the femur rod and tibia rod are to be connected together, the femur locking pin 24 is aligned with and moved into tibia socket 25, as shown in FIG. 5. The dimensions and shapes of the femur locking pin and the tibia socket are such that the tibia connector collar 23 is likely to become closely spaced from the femur connector collar 22.

The femur and tibia connector collars 22 and 23 provide opposed bearing surfaces 31 and 32 for use by the surgeon when the femur and tibia rods are to be connected, as illustrated in FIG. 5.

The femur and tibia rods 18 and 19 may be coated with an antibiotic of choice by the surgeon, as indicated at 29 and 30 of FIGS. 6 and 7. The antibiotic coatings 29 and 30 are applied to the femur and tibia stems 20 and 21, preferably with the coating being uniformly applied along the full length of the femur and tibia stems. The antibiotic coatings 29 and 30 may be applied to the femur and tibia rods before the rods are connected together, as illustrated in FIG. 6.

It is anticipated that the surgeon will select the femur and tibia rods of proper dimensions to fit the medullary cavities of the femur and tibia, even with the possibility that one medullary cavity might be of greater or lesser breadth than the other. Further, the femur and tibia rods are of smaller breadth than the anticipated breadth of the medullary cavities of the femur and tibia so that when an antibiotic coating 29 is applied, the antibiotic coating tends to fill the space within the medullary cavities not occupied by the stems 20 and 21 of the femur and tibia. This permits the antibiotic coating to substantially fill and contact the facing surfaces of the medullary cavities. The femur and tibia rods may have a treated surface that is adherent to polymethyl methacrylate (“PMMA”) and other medications that tend to cling to the femur and tibia rods 18 and 19.

As shown in FIG. 4, the exterior surface of the femur and tibia stems 20 and 21 are formed with irregular surfaces for the purpose of helping to retain the antibiotic coating 29 of FIG. 6. The irregular surfaces may be in the form of surface grooves 30 and 31 for the femur and tibia rods. This tends to stabilize the antibiotic/PMMA coating on the rods.

When the surgeon inserts the femur and tibia rods 18 and 19 in the medullary cavities of the femur and tibia, the rods are inserted separately, not when they are joined together in the medullary cavities. Once properly installed by moving the femur and tibia rods telescopically into the medullary cavities of the femur and tibia, the femur and tibia may be moved in alignment, causing the femur and tibia rods 18 and 19 to also become aligned, and the surgeon then can align the femur locking pin 24 with the tibia socket 25, so that the surgeon can grasp the femur and tibia connector collars 22 and 23 with an instrument that urges the connector collars toward one another with the femur locking pin 24 entering the tibia socket 25 with a friction fit. This may also be performed manually. It will be noted that the femur stem 25 is tapered, with the tibia socket having a complementary taper so that when the two are telescopically joined, a friction fit is formed between the femur locking pin 24 and the tibia socket 25. This is shown in the circled area of FIG. 6 and in the detailed area of FIG. 7.

Once the femur rod is joined with the tibia rod 19 as shown in FIGS. 6 and 7, the surgeon has the option to fill the space between the femur and tibia and around the connector collars 22 and 23 with antibiotic coating so that substantially the entire length of the temporary knee implant is coated with antibiotic coating.

The pin and socket connection 24, 25 maintains the femur rod and tibia rods in alignment with one another. This tends to freeze the femur and tibia of the leg of the patient in a fixed, extended position, tending to eliminate the bending of the leg so that a static connection is made.

When the sepsis of the knee joint has been eradicated, usually several weeks after the static connection has been made between the femur and tibia of the patient, the surgeon can use an appropriate tool to enter in the space 27 between the connector collars 22 and 23, and wedge or “pry” the femur and tibia connector collars apart. The femur lock pin 24 tends to withdraw axially from the tibia socket 25. When the surgeon bends the femur with respect to the tibia, the disconnected femur rod and tibia rod are then free to be withdrawn axially from their respective medullary cavities 15 and 16.

The type of antibiotic to be used by the surgeon can be selected to meet the needs of the patient. The antibiotic can be applied to the femur and tibia rods at the site of the surgery, or can be applied by the manufacturer/supplier of the femur and tibia rods, as desired by the surgeon.

While the surface of the femur and tibia rods is indicated as being formed by a series of indentations 30 and 31 at the exterior surfaces of the rods, other types of surface treatment may be used, such as but not limited to burling.

The procedure includes: first, the infected prosthesis is removed by the surgeon and the knee is thoroughly washed and debrided using standard technique. The femur and tibia are then reamed and further lavaged. The appropriate diameter/length rods are then coated with the PMMA/abx mixture or the prefabricated coated rods are brought into the field.

Once the coating has hardened, the rods are inserted separately into the femur and tibia with the knee flexed, and then the leg is extended until the tibia rod 19 is aligned with the femur rod 20 and the rods are locked centrally in alignment with each other. This will leave a space between the femur and the tibia that may be filled with a conventional PMMA/abx spacer that may be created by the surgeon or staff in the OR.

Some of the benefits of the procedure and apparatus are the patient cannot flex the knee, the rods are easily implanted and removed, and the customized antibiotics are directly applied to the infected area.

While this disclosure concerns a knee implant and procedure, this invention also may be applied to other joints of the human body.

Although a preferred embodiment of the invention has been disclosed in detail herein, it will be obvious to those skilled in the art that variations and modifications of the disclosed embodiment may be made without departing from the spirit and scope of the invention as set forth in the following claims.

1. A temporary knee implant to be placed at the position of the human knee joint for maintaining the tibia and femur at the knee joint aligned during healing of infection in the surrounding tissue, said knee prosthesis comprising:
an elongated femur rod including a femur stem having a proximal end and a distal end, said femur stem proximal end formed with a breadth sized and shaped to extend into the medullary cavity of the femur of a knee, and said elongated femur rod distal end including a femur connector.
an elongated tibia rod including a tibia stem having a proximal end and a distal end, said tibia stem distal end formed with a breadth sized and shaped to extend into the medullary cavity of the tibia, and said elongated tibia rod proximal end including a tibia connector.
said tibia connector and said femur connector including interfitting shapes for aligning said femur stem and said tibia stem.

2. The temporary knee implant of claim 1, wherein said
at least one of said femur stem and said tibia stem include an irregular exterior surface for retaining a coating.

3. The temporary knee implant of claim 2, wherein an antibiotic is applied to said irregular exterior surface of at least one of said femur rod and said tibia rod.

4. The temporary knee implant of claim 1, and further including a removable adhesive applied to said irregular surface of at least one of said femur stem and said tibia stem, so that the removable adhesive is positioned to adhere to the stem in the medullary cavity.

5. The temporary knee implant of claim 4, wherein said adhesive includes an antibiotic.

6. The temporary knee implant of claim 1, wherein said femur and tibia rods have a treated surface that is adherent to polymethyl methacrylate (“PMMA”).

7. The temporary knee implant of claim 1, wherein said femur connector and said tibia connector include interfitting surfaces for aligning the rods centrally with a space between said femur and said tibia for receiving PMMA/abx spacer,
said tibia connector and said femur connector including interfitting complementary shapes for aligning said femur stem and said tibia stem.

8. A temporary knee implant to be placed at the position of the human knee joint for maintaining the tibia and femur at the knee joint aligned during healing of infection in the surrounding tissue, said knee implant comprising:
an elongated femur rod including a femur stem having a proximal end and a distal end, said femur stem proximal end formed with a breadth sized and shaped to extend into the medullary cavity of the femur, and said elongated femur rod distal end including a femur connector,
an elongated tibia rod including a tibia stem having a proximal end and a distal end, said tibia stem distal end formed with of a breadth sized and shaped to extend into the medullary cavity of the tibia, and said elongated tibia rod proximal end including a tibia connector formed with a larger breadth than said tibia stem,
said tibia connector and said femur connector including interfitting shapes for aligning said femur stem and said tibia stem with space between said femur collar and said tibia collar sized for filling the space with medication.

9. The temporary knee implant of claim 8, wherein one of said femur connector and said tibia connector is formed with a locking mechanism and the other of said femur connector and said tibia connector formed with a shape to receive said locking mechanism and maintaining the femur and said tibia in spaced relationship for receiving filler material.

10. The temporary knee implant of claim 9, wherein said filler material is selected from the group consisting of adhesive, antibiotic, and PMMA/abx spacer material

11. A temporary knee implant to be placed at the position of the human knee joint for maintaining the tibia and femur at the knee aligned, comprising:
an elongated femur rod formed with a breadth sized and shaped to extend into the medullary cavity of the femur,
an elongated tibia rod formed with a breadth sized and shaped to extend into the medullary cavity of the tibia,
said tibia rod and said femur rod including interfitting shapes for connecting and aligning said femur rod and said tibia rod with a space between said femur rod and said tibia rod sized for receiving medicated spacer material.

12. The temporary knee prosthesis of claim 11, wherein said interfitting shapes are in the form of disks with one disk defining a socket and the other disk including a locking pin shaped to mate with said disk.

13. A process for maintaining joint alignment and spacing of a knee of the human body during recovery from infected knee prosthesis replacement, comprising:
removing the infected prosthesis,
washing the knee using standard medical technique,
coating a femur rod and a tibia rod with a PMMA/abx mixture and allowing time in which the mixture hardens.
inserting the rods separately into the femur and tibia with the knee flexed,
extending the knee until the rods are aligned,
locking the rods centrally with a space between the adjacent ends of the rods,
filling the space between the adjacent ends of the rods with a PMMA/abx spacer.
